(19) United States
(12) Patent Application Publication
(10) Pub. No.: US 2020/0282170 A1
(43) Pub. Date: Sep. 10, 2020

(54) OXYGEN CONCENTRATION DEVICE FOR ANESTHESIA MACHINE AND OXYGEN CONCENTRATION METHOD

(71) Applicant: Teruo KOBAYASHI, Yokohama-shi, Kanagawa (JP)

(72) Inventor: Teruo KOBAYASHI, Yokohama-shi, Kanagawa (JP)

(21) Appl. No.: 16/625,403

(22) PCT Filed: May 29, 2018
(86) PCT No.: PCT/JP2018/020562

§ 371 Date: Dec. 20, 2019

(30) Foreign Application Priority Data
Jul. 5, 2017 (JP) 2017-132120

(51) Int. Cl. A61M 16/10; B01D 53/04; A61D 7/04; A61M 16/00; A61M 16/01

(52) U.S. Cl. CPC A61M 16/101; B01D 53/0454; A61D 7/04; A61M 16/0063; A61M 16/01; A61M 16/022; A61M 2202/0208; B01D 2256/12; B01D 2257/102; B01D 2259/4533; A61M 2016/003; A61M 2250/00; B01D 2253/108

(57) ABSTRACT

An oxygen concentration device and an oxygen concentration method are capable of arbitrarily adjusting the concentration of an oxygen gas to be supplied to an anesthesia machine in a range of about 25 to 90% using an oxygen concentrator used for an ordinary oxygen treatment. An oxygen concentration device includes: a compressor configured to supply atmospheric air; oxygen concentration units configured to generate concentrated oxygen from the air supplied by the compressor; and a flow rate adjustment unit configured to purge a part of the concentrated oxygen suppliable from the oxygen concentration units to an anesthesia machine into the atmosphere, wherein the flow rate adjustment unit adjusts a flow rate of the concentrated oxygen to be purged into the atmosphere to control an oxygen concentration of the concentrated oxygen to be supplied to the anesthesia machine.

### TECHNICAL FIELD

The present invention relates to an oxygen concentration device used for a general anesthesia machine, for example, for a person, or an animal such as a cat or a dog, and an oxygen concentration method.

### BACKGROUND ART

Conventionally, an oxygen gas having a concentration of 100% has been used for general anesthesia using an anesthesia machine. However, in recent years, the adverse effect of anesthesia with pure oxygen is known, and an oxygen concentration of the oxygen gas to be inhaled of about 60% is regarded as preferable.

In order to supply the oxygen gas having an oxygen concentration of about 60% to a patient, a nitrogen gas or compressed air (sterile and low-humidity one) is separately prepared and mixed into oxygen to produce gas having an oxygen concentration of about 60%.

Further, when awakening from anesthesia at the finish of an operation, the oxygen concentration is further decreased, the patient is made to inhale oxygen having a low concentration close to the oxygen concentration of the atmosphere and adapts his or her body to the oxygen, thereby securing safety.

Patent document 1: International Publication No. WO 2016/098180

### DISCLOSURE OF THE INVENTION

### Problems to be Solved by the Invention

However, in the aforementioned method, it is necessary to separately prepare nitrogen or compressed air which is difficult to obtain on a daily basis and it is difficult to employ the method because of a problem in cost or the like at a site such as animal hospitals or the like.

Hence, in consideration of the above problem, an object of the present invention is to provide an oxygen concentration device and an oxygen concentration method capable of arbitrarily adjusting the concentration of an oxygen gas to be supplied to an anesthesia machine in a range of about 25 to 90% using an oxygen concentrator used for an ordinary oxygen treatment.

### Means for Solving the Problems

A first invention is an oxygen concentration device including: a compressor configured to supply atmospheric air; an oxygen concentration unit configured to generate concentrated oxygen from the air supplied by the compressor; and a flow rate adjustment unit configured to purge a part of the concentrated oxygen suppliable from the oxygen concentration unit to an anesthesia machine, into the atmosphere, wherein the flow rate adjustment unit adjusts a flow rate of the concentrated oxygen to be purged into the atmosphere to control an oxygen concentration of the concentrated oxygen to be supplied to the anesthesia machine.

In this case, it is preferable to further include a flowmeter capable of measuring the flow rate of the concentrated oxygen to be purged from the flow rate adjustment unit into the atmosphere.

A second invention is an oxygen concentration method including: a concentrated oxygen generation step of generating concentrated oxygen in an oxygen concentration unit from air supplied by a compressor; and a purge step of a flow rate adjustment unit purging (releasing) a part of the concentrated oxygen suppliable from the oxygen concentration unit to an anesthesia machine, into the atmosphere, wherein at the purge step, a flow rate of the concentrated oxygen to be purged (released) into the atmosphere is adjusted to control an oxygen concentration of the concentrated oxygen to be supplied to the anesthesia machine.

In this case, it is preferable that at the purge step, the flow rate of the concentrated oxygen to be purged (released) into the atmosphere is measurable by a flowmeter.

### Effect of the Invention

According to the present invention, the cost required for oxygen, nitrogen or compressed air used for the anesthesia machine can be significantly reduced and safe anesthesia can be executed and thus widely employed at the medical site.

### BRIEF DESCRIPTION OF DRAWINGS

FIG. 1 is configuration diagram of an oxygen concentration device of the present invention;

FIG. 2 is a graph illustrating a relationship between an oxygen flow rate and an oxygen concentration; and

FIG. 3 is a chart illustrating an experimental result of an oxygen concentration method of the present invention.

### BEST MODE FOR CARRYING OUT THE INVENTION

An oxygen concentration device and an oxygen concentration method according to an embodiment of the present invention will be explained.

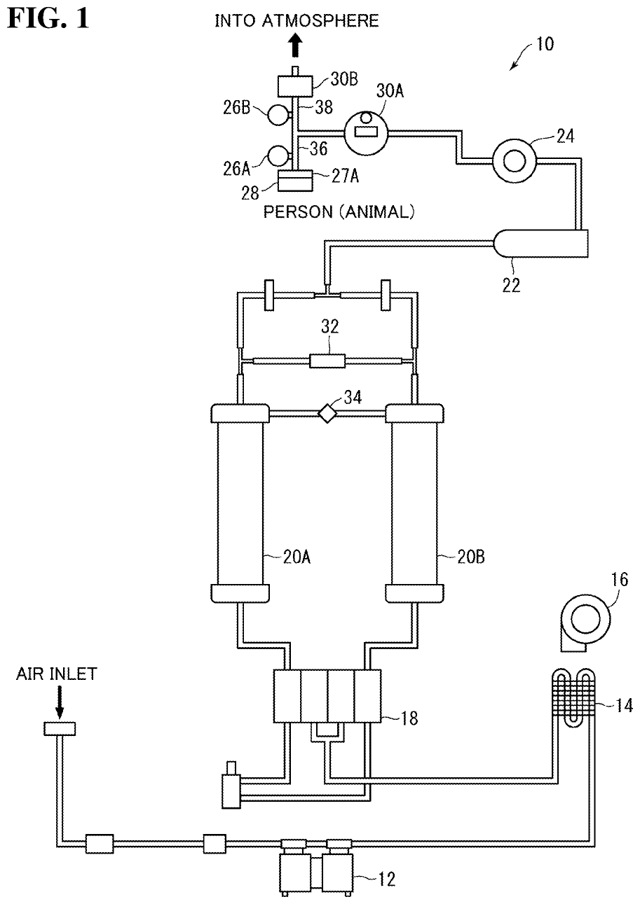

As illustrated in FIG. 1, an oxygen concentration device 10 includes mainly a compressor 12, a heat exchanger 14, a fan 16, an air controller 18, a pair of oxygen concentration units 20A, 20B, a concentrated oxygen tank 22, a pressure adjustment unit 24, a plurality of flowmeters 26A, 26B, and a plurality of flow rate adjustment units 30A 30B.

The compressor 12 is a supply source which supplies atmospheric air to the downstream side.

The heat exchanger 14 is intended to lower the temperature of compressed air which has been compressed by the compressor 12 and reached high temperature. When the fan 16 is driven, the air-cooled heat release effect can be obtained.

For the air controller 18, for example, a through valve manifold is employed. For the through valve manifold, four through valves are combined, and two valves are operated in each cycle to send air and exhaust nitrogen. The operation of the through valve manifold repeats a first cycle and a second cycle in a period of a predetermined time or a predetermined pressure. The operation cycle differs depending on the power supply frequency and the flow rate.

The pair of oxygen concentration units 20A, 20B each include, for example, a casing and zeolite housed in the casing. This is also called a molecular sieve and has an ability of separating molecules depending on the size of the molecule. Zeolite absorbs nitrogen and allows oxygen to pass therethrough. The reason is that the size of the nitrogen molecule is relatively large and is thus captured in the hole of zeolite, whereas the size of the oxygen molecule is relatively small and is thus not captured in the hole of zeolite. As a result of this, when the compressed air passes through zeolite, oxygen and nitrogen in the air are separated, so that nitrogen is absorbed in zeolite and concentrated oxygen is generated.

The generated concentrated oxygen is stored in the concentrated oxygen tank 22, and controlled by the pressure adjustment unit 24 to a predetermined value, and then branched at an outlet of the flow rate adjustment unit 30A.

In other words, on the outlet side of the flow rate adjustment unit 30A, two routes such as a first flow path 36 and a second flow path 38 are provided. An anesthesia machine 28 is connected to the first flow path 36, and the second flow path 38 is opened to the atmosphere via the other flow rate adjustment unit 30B.

Here, the relationship between the oxygen concentration and the flow rate will be considered.

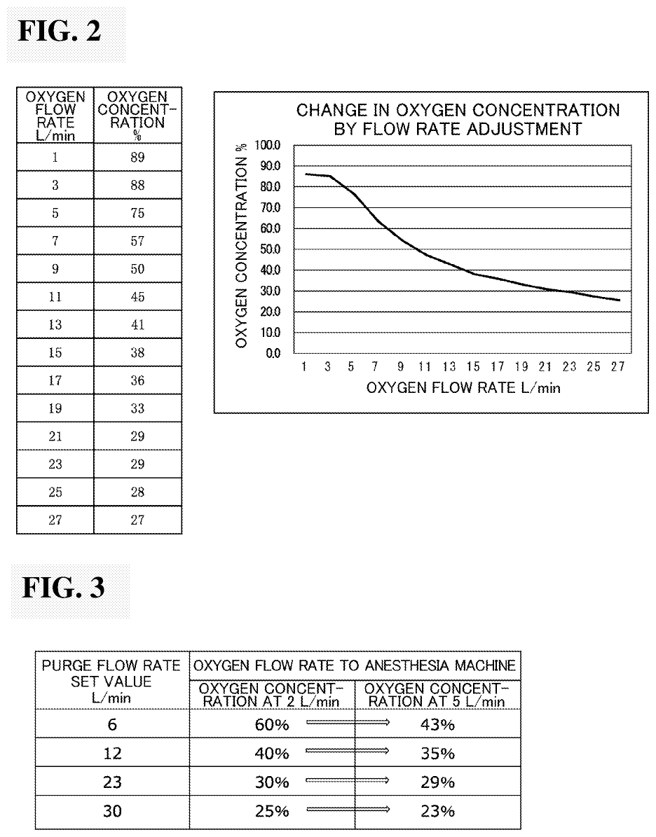

As illustrated in FIG. 2, when an oxygen concentration device which makes the oxygen concentration about 90% at a flow rate at the time when supplying the concentrated oxygen is, for example, 2 L/min is used, the oxygen concentration becomes about 25% by adjusting the flow rate to 30 L/min. Therefore, to solve the problem in supplying highly-concentrated oxygen to a patient, it is only necessary to supply the concentrated oxygen to the anesthesia machine 28 at a flow rate as high as possible.

However, if the concentrated oxygen is supplied to the anesthesia machine 28 at a high flow rate, concentrated oxygen containing an anesthesia component other than the concentrated oxygen consumed by the patient is simultaneously exhausted in the operating room, possibly resulting in interference with an appropriate medical practice by a doctor, medical staff and so on.

From the above reason, it is necessary to avoid the supply of the concentrated oxygen at a flow rate more than necessary to the anesthesia machine 28.

The anesthesia machine 28 is intended to provide anesthetic effects to the patient, and the concentrated oxygen at a predetermined concentration is supplied to the anesthesia machine 28. The flowmeter 26A and a flow rate adjustment unit 27A are provided closely to or integrally with the anesthesia machine 28 and can adjust the flow rate of the concentrated oxygen to be supplied to the anesthesia machine 28.

The flow rate adjustment unit 30B of the second flow path 38 is similarly provided with the flowmeter 26B.

Note that the flow rate adjustment units 30A, 30B adjust the flow rates of gas flowing through the flow paths.

Next, the operation of the oxygen concentration device and the oxygen concentration method according to the embodiment will be explained.

As illustrated in FIG. 1, the concentrated oxygen is stored in the concentrated oxygen tank 22. A part of the concentrated oxygen in the concentrated oxygen tank 22 is supplied to the first flow path 36, and the residual of the concentrated oxygen is supplied to the second flow path 38.

The concentrated oxygen supplied to the first flow path 36 is supplied to the patient via the anesthesia machine 28 while the flow rate is being appropriately adjusted by the flow rate adjustment unit 27A.

The concentrated oxygen supplied to the second flow path 38 is purged into the atmosphere while the flow rate is being appropriately adjusted by the flow rate adjustment unit 30B.

Here, the relationship between the flow rate and the concentration of the concentrated oxygen is illustrated in FIG. 2 as the characteristics of the oxygen concentration device 10. It is illustrated that as the flow rate of the oxygen concentration device 10 is increased, the oxygen concentration decreases.

As explained above, when the concentrated oxygen having a high concentration of, for example, 90% is taken into the body of the patient for a long time, the concentrated oxygen conversely generates a harmful effect. Therefore, it has been necessary to increase the flow rate of the concentrated oxygen to decrease the concentration of the concentrated oxygen to be supplied to the patient.

However, at the time when the flow rate of the concentrated oxygen is is increased, the concentrated oxygen which has not been consumed by the patient but remains of the concentrated oxygen containing the anesthesia component is released into the operating room via a relief valve installed in the anesthesia machine 28 to lead to a risk of interference with the medical practice by the medical personnel.

Therefore, a part of the concentrated oxygen to be supplied from the concentrated oxygen tank 22 is intentionally made to pass through the second flow path 38 and purged into the atmosphere not via the anesthesia machine 28, thereby making it possible to simultaneously reduce the flow rate and the oxygen concentration of the concentrated oxygen to be supplied to the patient through the first flow path 36 and via the anesthesia machine 28.

In the present invention, because the oxygen concentration of an oxygen gas to be supplied to the anesthesia machine is diluted, compressed air and nitrogen gas become unnecessary.

Note that a code 32 denotes an orifice, and a code 34 denotes a PE valve.

Next, an experimental example of the oxygen concentration method in the embodiment will be explained.

An experiment for confirming how the oxygen concentration of the concentrated oxygen supplied to the anesthesia machine 28 changes according to the change in flow rate value of the concentrated oxygen purged into the atmosphere by adjusting the flow rate adjustment unit 30B illustrated in FIG. 1 was carried out.

In this experiment, as illustrated in FIG. 3, the flow rate of the concentrated oxygen supplied to the anesthesia machine 28 was changed at 2 L/min and 5 L/min. Further, the flow rate of the concentrated oxygen supplied from the second flow path 38 into the atmosphere was changed at 6 L/min, 12 L/min, 23 L/min, and 30 L/min.

As illustrated in FIG. 3, at the flow rate of the concentrated oxygen purged from the second flow path 38 into the atmosphere of 6 L/min, the oxygen concentration of the concentrated oxygen was 60% in the case where the flow rate of the concentrated oxygen supplied to the anesthesia machine 28 was 2 L/min and the oxygen concentration of the concentrated oxygen was 43% in the case where the flow rate was 5 L/min.

At the flow rate of the concentrated oxygen purged from the second flow path 38 into the atmosphere of 12 L/min, the oxygen concentration of the concentrated oxygen was 40% in the case where the flow rate of the concentrated oxygen supplied to the anesthesia machine 28 was 2 L/min and the oxygen concentration of the concentrated oxygen was 35% in the case where the flow rate was 5 L/min.

At the flow rate of the concentrated oxygen purged from the second flow path 38 into the atmosphere of 23 L/min, the oxygen concentration of the concentrated oxygen was 30% in the case where the flow rate of the concentrated oxygen supplied to the anesthesia machine 28 was 2 L/min and the oxygen concentration of the concentrated oxygen was 29% in the case where the flow rate was 5 L/min.

At the flow rate of the concentrated oxygen purged from the second flow path 38 into the atmosphere of 30 L/min, the oxygen concentration of the concentrated oxygen was 25% in the case where the flow rate of the concentrated oxygen supplied to the anesthesia machine 28 was 2 L/min and the oxygen concentration of the concentrated oxygen was 23% in the case where the flow rate was 5 L/min.

It was turned out from the above result that the relationship between the flow rate of the concentrated oxygen supplied to the anesthesia machine 28 and the oxygen concentration was that when the flow rate of the concentrated oxygen supplied to the anesthesia machine 28 was increased (2 L/min→5 L/min), the oxygen concentration of the concentrated oxygen decreased by the value of the flow rate of the concentrated oxygen purged from the second flow path 38 into the atmosphere. This has proved that when the flow rate of the concentrated oxygen to be supplied to the anesthesia machine 28 is increased, the oxygen concentration of the concentrated oxygen decreases.

It has been further proved that when the value of the flow rate of the concentrated oxygen to be purged from the second flow path 38 into the atmosphere is increased, the oxygen concentration of the concentrated oxygen to be supplied to the anesthesia machine 28 decreases. This has proved that the value of the flow rate of the concentrated oxygen to be supplied to the anesthesia machine 28 has the same tendency at 2 L/min and 5 L/min.

However, it has been proved that when the value of the flow rate of the concentrated oxygen to be purged from the second flow path 38 into the atmosphere is increased, the difference in the oxygen concentration of the concentrated oxygen to be supplied to the anesthesia machine 28 decreases between the magnitudes (values at 2 L/min and 5 L/min) of the flow rate of the concentrated oxygen to be supplied to the anesthesia machine 28. In other words, it has been turned out that when the flow rate value of the concentrated oxygen to be purged from the second flow path 38 into the atmosphere is increased, the oxygen concentration of the concentrated oxygen to be supplied to the anesthesia machine 28 tends to converge to decrease to a certain value irrespective of the value of the flow rate of the concentrated oxygen to be supplied to the anesthesia machine 28.

As explained above, it has been turned out that the purge of the concentrated oxygen from the anesthesia machine 28 into the atmosphere greatly contributes to a decrease in the oxygen concentration of the concentrated oxygen to be supplied to the anesthesia machine 28.

It has been turned out, on the above premise, that when the flow rate of the concentrated oxygen to be supplied to the anesthesia machine 28 is increased, the oxygen concentration of the concentrated oxygen to be supplied to the anesthesia machine 28 decreases irrespective of the magnitude of the flow rate value of the concentrated oxygen to be purged from the second flow path 38 into the atmosphere.

It has been further turned out that when the flow rate value of the concentrated oxygen to be purged from the second flow path 38 into the atmosphere is increased, the oxygen concentration of the concentrated oxygen to be supplied to the anesthesia machine 28 converges to decrease to a certain value irrespective of the magnitude of the flow rate value of the concentrated oxygen to be supplied to the anesthesia machine 28.

### EXPLANATION OF CODES

10 oxygen concentration device
12 compressor
14 heat exchanger
16 fan
18 air controller
20A oxygen concentration unit
20B oxygen concentration unit
22 concentrated oxygen tank
24 pressure adjustment unit
26A flowmeter
26B flowmeter
27A flow rate adjustment unit
28 anesthesia machine
30A flow rate adjustment unit
30B flow rate adjustment unit
32 orifice
34 PE valve
36 first flow path
38 second flow path

1. An oxygen concentration device comprising:
a compressor configured to supply atmospheric air;
an oxygen concentration unit configured to generate concentrated oxygen from the air supplied by the compressor; and
a flow rate adjustment unit configured to purge a part of the concentrated oxygen suppliable from the oxygen concentration unit to an anesthesia machine, into the atmosphere,
wherein the flow rate adjustment unit adjusts a flow rate of the concentrated oxygen to be purged into the atmosphere to control an oxygen concentration of the concentrated oxygen to be supplied to the anesthesia machine.

2. The oxygen concentration device according to claim 1, further comprising a flowmeter capable of measuring the flow rate of the concentrated oxygen to be purged from the flow rate adjustment unit into the atmosphere.

3. An oxygen concentration method comprising:
a concentrated oxygen generation step of generating concentrated oxygen in an oxygen concentration unit from air supplied by a compressor; and
a purge step of a flow rate adjustment unit purging a part of the concentrated oxygen suppliable from the oxygen concentration unit to an anesthesia machine, into the atmosphere,
wherein at the purge step, a flow rate of the concentrated oxygen to be purged into the atmosphere is adjusted to control an oxygen concentration of the concentrated oxygen to be supplied to the anesthesia machine.

4. The oxygen concentration method according to claim 3, wherein at the purge step, the flow rate of the concentrated oxygen to be purged into the atmosphere is measurable by a flowmeter.
